(19) United States
(12) Patent Application Publication
(10) Pub. No.: US 2026/0013833 A1
(43) Pub. Date: Jan. 15, 2026

(54) PREOPERATIVE METHOD AND SYSTEM FOR MINIMIZING WOUND COMPLICATIONS

(71) Applicants: John T. Lindsey, Metairie, LA (US); Chris Spring, New Orleans, LA (US)

(72) Inventors: John T. Lindsey, Metairie, LA (US); Chris Spring, New Orleans, LA (US)

(21) Appl. No.: 19/334,202

(22) Filed: Sep. 19, 2025

Related U.S. Application Data
(63) Continuation-in-part of application No. 18/198,697, filed on May 17, 2023, now Pat. No. 12,419,612.
(60) Provisional application No. 63/407,939, filed on Sep. 19, 2022.

(51) Int. Cl. A61B 8/00; A61B 8/08; G06N 3/0464; G06N 3/09; G06T 7/00

(52) U.S. Cl. CPC A61B 8/5223; A61B 8/0858; G06N 3/0464; G06N 3/09; G06T 7/0012; G06T 2207/10132; G06T 2207/20081; G06T 2207/30004

(57) ABSTRACT

A system and method for preoperatively predicting wound complications and recommending tension reducing procedures is disclosed. The system includes (i) ultrasound imaging technology operable to take an ultrasound of a portion of subcutaneous tissue on a patient, (ii) image processing and filtering technology operable to focus on a portion of the tissue and filter out the overlying dermis, underlying muscle, and muscle fascia, and (iii) processing means capable of determining the Mean Gray Value (MGV) from the imaged sample. The method further includes tension reducing procedures for patients with a MGV less than 0.127 to minimize foreseeable wound complications. The system may include a processor and image classification engine operable to classify any ultrasound image and determine the MGV from the imaged sample.

### RELATED APPLICATIONS

This application is a continuation in part application of the U.S. patent application Ser. No. 18/198,697, filed May 17, 2023, which claims priority to U.S. Provisional Application No. 63/407,939, filed Sep. 19, 2022. The entire contents of the above applications are hereby incorporated by reference as though fully set forth herein.

### FIELD OF TECHNOLOGY

The present invention relates in general to preoperative medical procedures, and in particular to a system and method for minimizing wound complications during surgery.

### BACKGROUND

Dramatic weight loss has many benefits. But after any substantial amount of weight loss due to weight loss surgery and/or lifestyle changes, the skin and tissues often lack the elasticity to conform to the reduced body size. Surgical body contouring following major weight loss, pregnancy, or because of the normal ageing process, removes excess sagging skin and fat while restoring or improving the shape of the body. The result is a better-proportioned appearance, smoother contours, and often improved functionality. As a result, the demand for body contouring surgery continues to rise.

One increasingly popular procedure that enhances the functional and aesthetic outcomes in this population is that of abdominoplasty. According to the American Society of Plastic Surgeons, the number of abdominoplasties has risen 107 percent since 2000, up to 130,081 procedures in the United States in 2018. Other body contouring procedures such as brachioplasty and thighplasty have likewise increased in the United States and worldwide in a similar fashion. Although popularity of plastic surgery is on the rise, wound complications reported as high as 51.8 percent in bariatric patients have plagued these procedures.

With these procedures in high demand, there is a need to address the high percentage of wound complications associated with these procedures. It is an object of this invention to minimize wound complications by identifying the Mean Gray Value (MGV) of tissue at the surgical site to determine if certain tension reducing procedures should be recommended prior to surgery. In addition, the system and method for determining MGV herein can be applied more broadly to benefit patients with potential wound complications. For example, while wound closure technology has improved, such devices (e.g. negative-pressure vacuum device) are burdensome, cumbersome, and very expensive. The disclosed invention could be utilized to vet ideal candidates who would benefit from these devices for procedures where postural or position changes for reducing tension at the wound site are not available.

### BRIEF SUMMARY OF THE INVENTION

In a first embodiment, a system and method for predicting wound complications is disclosed. The system includes (i) ultrasound imaging technology operable to take an ultrasound of a portion of subcutaneous tissue on a patient, (ii) image processing and filtering technology operable to focus on a portion of the tissue and filter out the overlying dermis, underlying muscle, and muscle fascia, and (iii) processing means capable of determining the Mean Gray Value (MGV) from the imaged sample. Based on the MGV value, a reliable prediction of wound complications can be provided to the patient as well as recommendations for tension-reducing procedures to minimize foreseeable wound complications. Such procedures involve skin excision and advancement, while ensuring appropriate and consistent patient positioning and avoidance of wound tension and skin pleating in at-risk patients. Alternatively, if posture and position adjustments are not available to reduce tension, the MGV value may be utilized to make a recommendation for a suitable wound closure device, including a negative-pressure vacuum device (e.g. negative pressure wound therapy device manufactured by PICO, Prevena, KCI Wound VAC, Renasys, and Solventum).

In a second embodiment, the system comprises a processing module including a processor and image classification engine having a neural network trained to classify any ultrasound image into the following sets of data labels: (i) type of ultrasound image, (ii) size of the ultrasound image, (iii) size of the target area of interest (i.e. portion of the tissue after filtering out the overlying dermis, underlying muscle, and muscle fascia), and (iv) annotated descriptor for the region of the human body being examined. The processing module is operable to determine the MGV of the target area of interest to predict wound complications.

A third embodiment includes the method for training an image classification engine comprising a neural network model comprising the steps of (i) providing a training dataset of ultrasound images to the neural network with labels corresponding to each data label described above, (ii) receiving a predicted output, (iii) adjusting the weights of the neural network to minimize the difference between the predicted output and the actual label of each image in the training dataset, and (iv) repeating the above steps until the neural network can predict the category of new, unseen images with a high degree of accuracy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow diagram showing a method for predicting wound complications preoperatively in accordance with the embodiments of the present invention.

FIG. 2 is a flow diagram showing a method for training a neural network in accordance with the embodiments of the present invention.

FIG. 3A is an ultrasound image showing the pubic area of the lower anterior abdominal wall in a patient undergoing abdominoplasty. The cropped image, as indicated by the rectangle, excludes the overlying dermis and underlying muscle and muscle fascia.

FIG. 3B is an ultrasound image showing the pubic area of the lower anterior wall in a different patient undergoing abdominoplasty. The cropped image, as indicated by the rectangle, excludes the overlying dermis and underlying muscle and muscle fascia.

FIG. 4 is a diagram depicting a tension reducing procedure in accordance with embodiments of the invention by showing a cross-section view of human skin with skin flap margins in apposition after measured resection prior to final layered wound closure.

FIG. 5A is a diagram that shows pleating along a lateral abdominoplasty incision.

FIG. 5B is a diagram depicting a tension reducing procedure in accordance with embodiments of the invention by showing an incision that has been extended laterally to make-up skin length mismatch and to avoid pleating of the skin.

FIG. 6A is a diagram depicting a tension reducing procedure in accordance with embodiments of the invention by showing a thigh lift skin excision planned and measured with thighs in 30 degrees of abduction for patients.

FIG. 6B is a diagram that shows a thigh lift skin excision incorrectly planned and measured with the thighs fully adducted for patients.

FIG. 7A is a diagram depicting a tension reducing procedure in accordance with embodiments of the invention by showing an abdominoplasty skin excision planned and measured with waist flexed at 10 degrees or less for patients.

FIG. 7B is a diagram that shows an abdominoplasty skin excision incorrectly planned and measured with typical 40 degree waist flexion for patients.

FIG. 8 is a flow diagram showing a system for predicting wound complications preoperatively in accordance with the embodiments of the present invention.

FIG. 9 is an ultrasound image used for the image classification engine in accordance with embodiments of the present invention.

FIG. 10 is a cropped view of a portion of the ultrasound image depicted in FIG. 9 showing the target area of interest.

FIG. 11 is an ultrasound image showing a doctor's annotation for the depth or size of the target area of interest.

### DETAILED DESCRIPTION

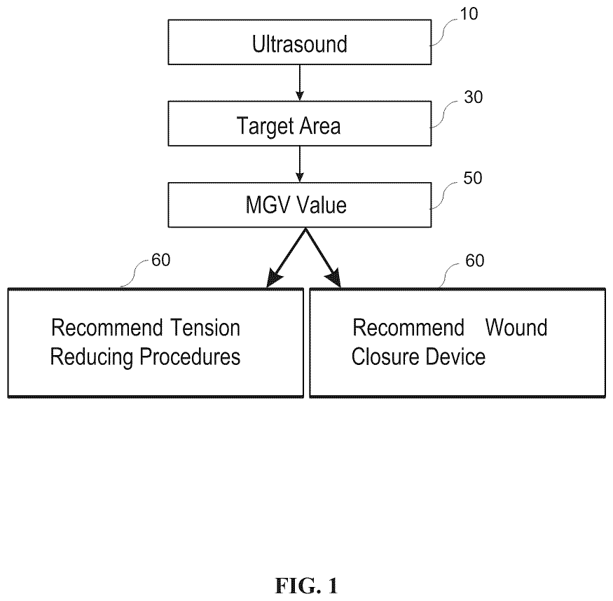

A system and method for preoperatively predicting wound complications for a patient undergoing body contouring surgery is shown in FIG. 1. The first step 10 in the method requires the use of ultrasound imaging technology to take an ultrasound image of the portion of the patient's body where the surgery is to take place. In one embodiment, this step is accomplished using, for example, a Lumify portable ultrasound system which allows the physician to view the subcutaneous tissue. This imaging is preferably accomplished in B mode on a Samsung Galaxy Tab A tablet on the superficial setting, gain set to 54 and depth settings between 2.5-4 cm, depending on the thickness of the subcutaneous tissue. An example ultrasound image 20 taken using the Lumify portable ultrasound system is shown in FIG. 11. Here, the attending physician has included an annotation corresponding to the depth of interest for the subcutaneous tissue. This annotation may be manually applied by the physician using the ultrasound machine.

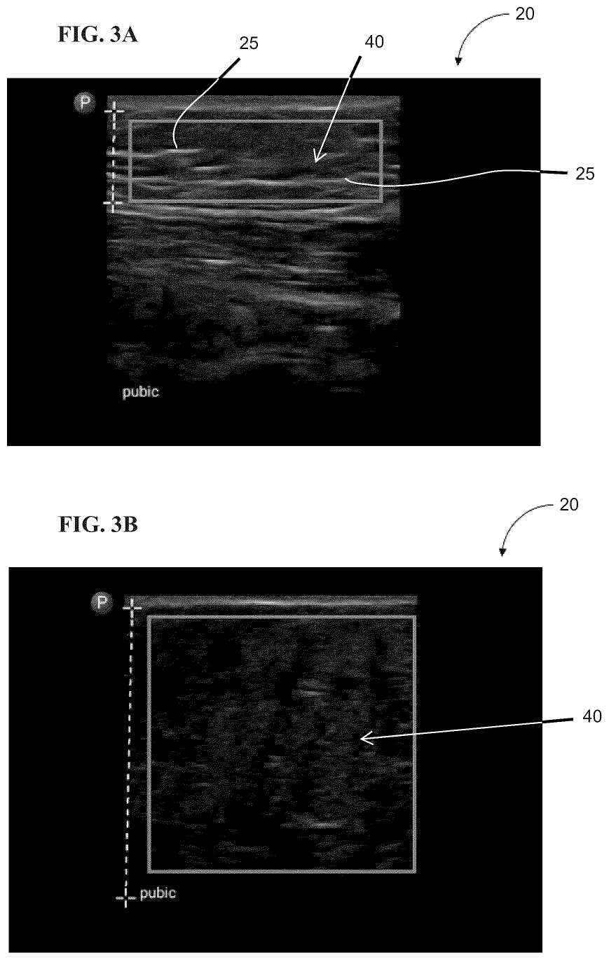

FIGS. 3A-3B show two separate ultrasound images for patients undergoing an abdominoplasty. The second step 30 in the method is to crop the image 20 to focus on the target area 40 (as shown in FIGS. 3A-3B) of the image 20 by excluding the overlying dermis and underlying muscle and muscle fascia. This step is accomplished using, for example, XnView, or any equivalent software. As shown in FIGS. 3A and 3B, the portions of the ultrasound image 20 within the rectangles demonstrate the target areas 40 of subcutaneous tissue cropped by XnView.

The third step 50 in the method includes analyzing the cropped image to determine the MGV of the ultrasound image 20. This step is accomplished using, for example, CellProfiler, or its equivalent, to determine the MGV of the sample based on the following equation:

$$\begin{matrix}Mean Gray Volume = \frac{\sum Echogenicity of Each Pixel}{Number of Pixels in Image} \\ = \frac{Total Echogenicity of Image}{Area}\end{matrix}$$

As shown in FIG. 3A, the image 20 includes multiple horizontally-oriented streaks of white, reflective collagen 25 that indicate a strong SFS (superficial fascial system) in this area with a corresponding MGV of 0.16296. Conversely, the image depicted in FIG. 3B shows the subcutaneous tissue almost devoid of collagen 25 which indicates a weak SFS with a corresponding MGV of 0.06206.

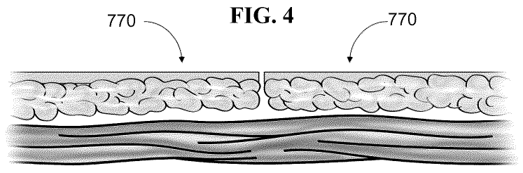

In the fourth step 60, patients with average to poor MGV (0.127 or less) are identified preoperatively for recommended tension-reducing procedures to reduce wound complications before undergoing a specific type of body contouring procedure. The primary purpose of these tension reducing procedures is to avoid tension of the skin during wound closure, which is a common cause of wound complications. The recommended clinical maneuvers undertaken to reduce tension closure in body contouring surgery are depicted in FIGS. 4, 5B, 6A, and 7A, each of which are summarized below. Alternatively, if posture and position adjustments are not available to reduce tension, the MGV value may be utilized to make a recommendation for a suitable wound closure device, including a negative-pressure vacuum device (e.g. negative pressure wound therapy device manufactured by PICO, KCI Wound VAC, Prevena, Renasys, and Solventum).

As shown in FIG. 4, each tension reducing procedure involves removing excess skin so that cut skin flaps 70 lay in apposition rather than gap apart. Closing a gap in the skin during body contouring provides improved appearance and contour but at a risk of wound-healing complications. There should be no gap in the skin flaps at closure for patients with MGV<0.127.

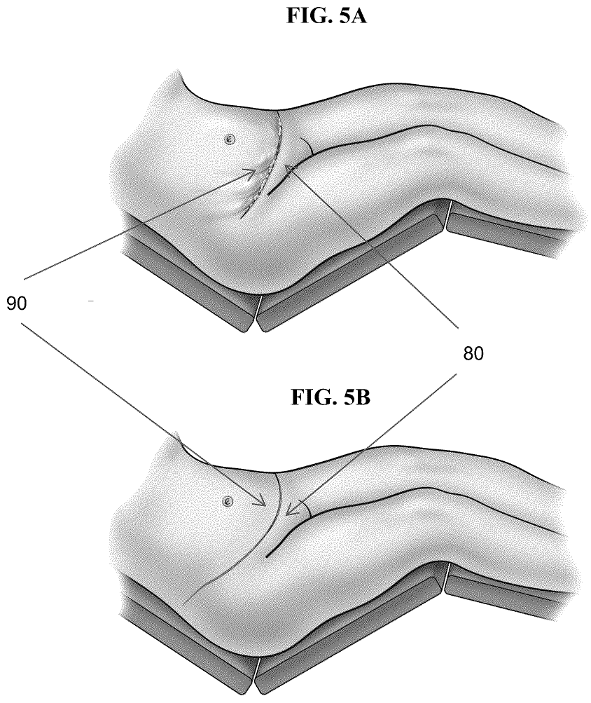

Another source of wound complications is skin pleating, as there may be irregular and uneven skin margin match at the closure site. This lack of smooth and even skin flap coaptation decreases the wound healing area of contact. The image depicted in FIG. 5A shows post-closure pleating of the skin due to skin length mismatch during an abdominoplasty. This occurs when the length of the more cephalic incision for skin excess is longer than the length of the skin incision for the caudal skin excess. This is a common scenario on many parts of the body where the girth of one body part (i.e., mid-abdominal area) exceeds the girth of another area (hip area), and the intervening skin excess requires removal. In contrast, the image in FIG. 5B avoids skin pleating and demonstrates the recommended tension reducing procedure. As shown, a smooth and uniform wound closure ensues, thus maximizing wound healing contact area of the skin flaps. This is achieved by lengthening the caudal abdominoplasty incision laterally. By doing so, the skin length mismatch between the shorter caudal incision 80 and the longer cephalic incision 90 is averaged over a longer distance, such that pleating of the cephalic skin flap can be progressively diminished until smooth, and maximal soft tissue contact can be achieved during wound closure.

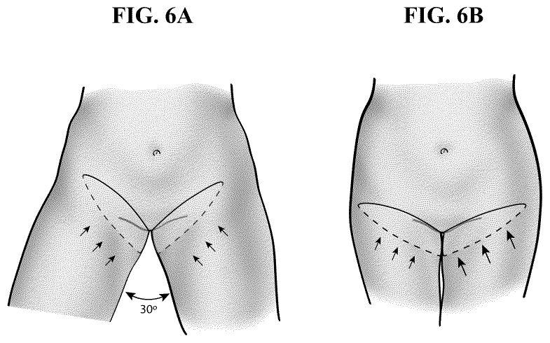

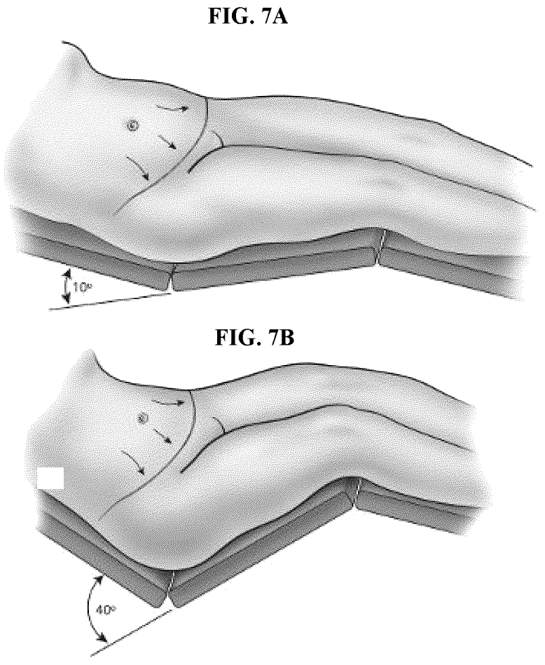

The other tension reducing procedure includes the avoidance of postural body changes. For patients undergoing the body contouring procedure of a thigh lift closure, for example, the recommended tension reducing procedure for at-risk patients with MGV<0.127 includes skin resection done with skin apposition at 30 degrees of thigh abduction (FIG. 6A) rather than with the thighs fully adducted (FIG. 6B)—the latter of which results in increased tightening and tension and should be done for patients with MGV>0.127. For patients undergoing abdominoplasty with an MGV<0.127, the recommended tension-reducing procedure includes waist flexion limited to no more than 10 degrees as part of “beach chair” positioning, as shown in FIG. 7A. For patients with MGV>0.127, waist flexion as much as 40 degrees (FIG. 7B) is routine. Avoidance of body postural changes for patients with MGV<0.127 decreases wound healing complications.

A study has demonstrated that this method has proven successful in reducing wound complications when compared to a retrospective cohort. As shown in the table below, the cohorts were similar except for a higher incidence of diabetes in the retrospective group (1 v 9, p=0.026, table 1).

| | Prospective | Retrospective | p-value |
|---|---|---|---|
| Age (yrs) | 45.9 | 47.6 | 0.313 |
| BMI | 29.2 | 28.1 | 0.083 |
| Weight Resected (gr) | 1045.6 | 1180.4 | 0.450 |
| Diabetes | 1 | 9 | 0.026 |
| Smoking | 1 | 0 | — |
| Hx Massive Weight Loss | 4 | 8 | 0.254 |
| Hx Bariatric Surgery | 23 | 30 | 0.323 |
| Wound Complications | 5 | 19 | 0.006 |
| Major Wound Complications | 0 | 1 | 0.978 |
| Total Patients | 112 | 115 | |

The wound complication rate was significantly reduced in the prospective group (5/112, 4.4%) when compared to the retrospective group (20/115, 17%, p=0.0062).

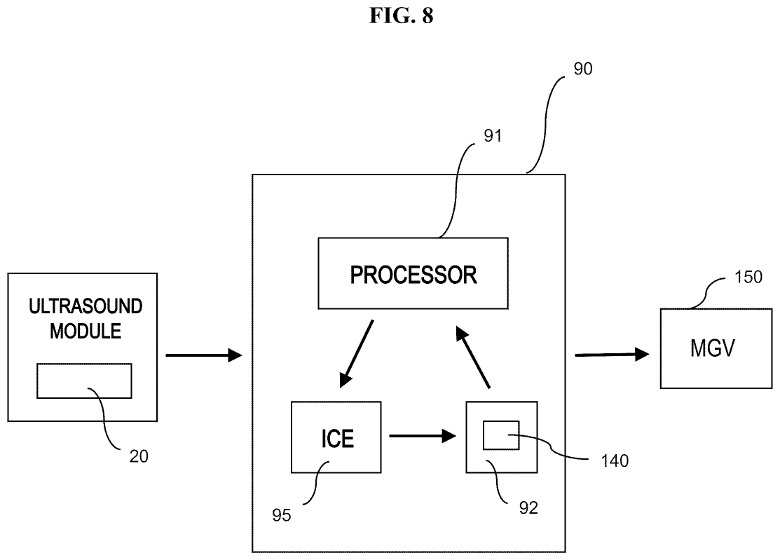

Turning to FIG. 8, an alternative embodiment of the invention includes a processing module 90 operable to receive an ultrasound image 20 from an ultrasonic machine. FIG. 11 is an example ultrasound image that has been taken of a patient's subcutaneous tissue. The attending physician may annotate the depth or size of the target area of interest 120 on the ultrasound imaging machine before it is exported to the processing module 90. Alternatively, the image classification engine may be operable to identify the size of the target area of interest 120.

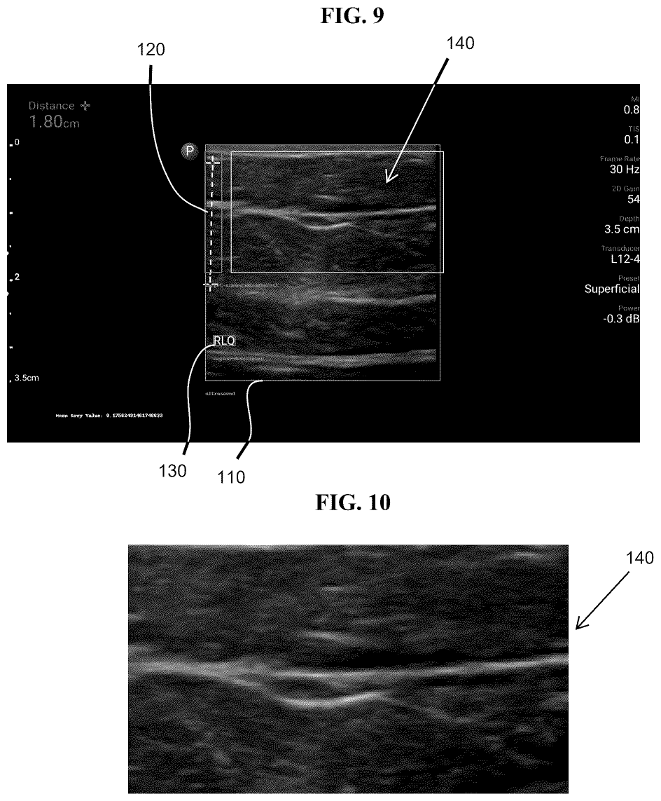

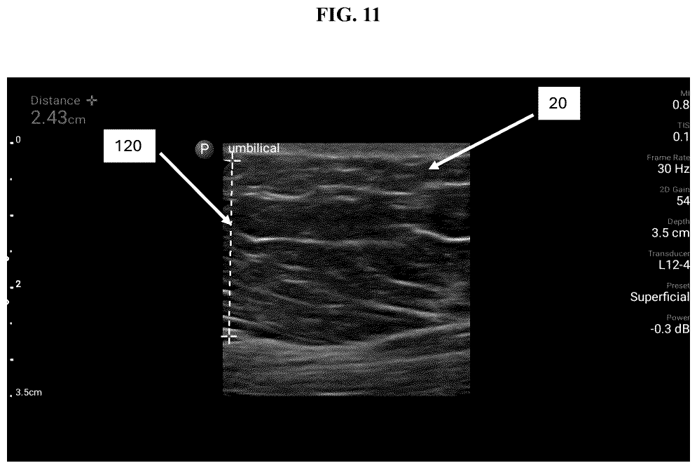

The processing module 90 includes a processor 91 and an image classification engine 95 that utilizes a convolutional neural network specifically trained to receive an ultrasound image 20 and apply the following labels of data within the ultrasound image 20: (i) type of ultrasound image 100 (not shown), (ii) size of the ultrasound image 110, (iii) size of the target area of interest (i.e. portion of the tissue and filter out the overlying dermis, underlying muscle, and muscle fascia) 120, and (iv) annotated descriptor for the region of the human body being examined 130. An example of the ultrasound image 20 with these labels is show in FIG. 9. The processing module 90 extracts an annotated export image 92 identifying the target area of interest 140 in the image 20 and the associated pixel values. Based on these outputs of data, the processing module 90 can determine the MGV 150 for the target area of interest 140 (as shown in FIG. 10).

An exemplary embodiment for the image classification engine is a YOLO (You Only Look Once) model, however, other types of object detection models can be utilized (e.g. SSD, Faster R-CNN, or RetinaNet). The YOLO model may be implemented using other tools, including for example, YOLOv8 which utilizes an open-source framework such as PyTorch or TensorFlow.

The architecture for the image classification engine 95 includes an input layer, a backbone network, neck, and detection head. The input layer resizes the ultrasound image to be run against the neural network model to 640×640 pixels with letterboxing to maintain the original aspect ratio without distortion. After the ultrasound image is resized, the backbone network is configured to extract hierarchical convolutional features for image analysis. Exemplary backbone networks include CSPDarkent or EfficientNet. The neck is operable to combine features from different layers to detect objects of various sizes. The neck may utilize a Path Aggregation Network (PANet) or similar multi-scale feature fusion technique to integrate high and low-level layers for accurate detection of both large (ultrasound scan regions) and smaller (annotation markets) elements within images. The detection head predicts bounding box coordinates with associated class labels and confidence scores for the bounding box encompassing the active ultrasound scan area and the bounding box for the target area 140.

In operation, the image classification engine receives an ultrasound image frame as a digital array of pixel intensities. The input layer of the engine resizes the image into a standardized resolution, which for the preferred embodiment is 640×640 pixels. The image classification engine divides the grid into an S×S grid of equal sized cells. Each individual grid cell is responsible for detecting objects whose center falls within its boundaries. The convolutional backbone network applies multiple convolutional filters across the image to create hierarchical feature maps at different scales, including high-resolution maps (retain fine spatial detail but shallow semantics) and low-resolution maps (capture global semantic information but with coarse detail). The neck of the neural network model architecture may access a Path Aggregation Network to integrate high and low resolution maps to provide precise bounding box regression and classification. The Path Aggregation Network then outputs multi-scale maps to the detection head which predicts bounding boxes for the ultrasound image with associated class labels and confidence scores.

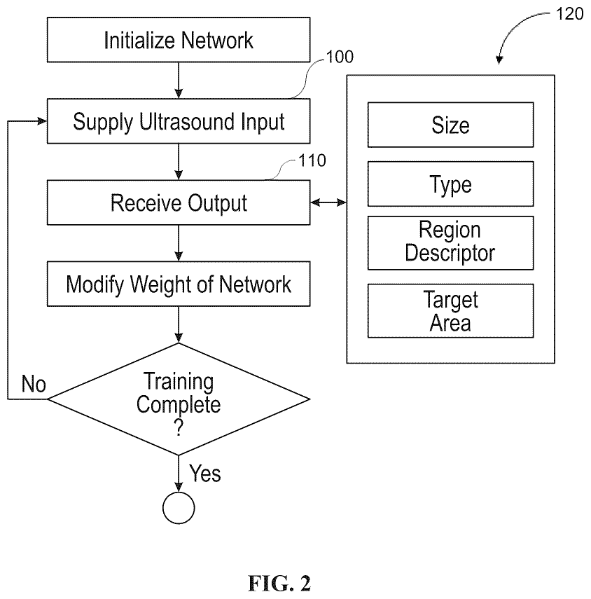

Turning to FIG. 2, the method utilized to train the neural network of the image classification engine 95 is disclosed. The neural network is trained on an input dataset of ultrasound images 100 that have been labeled as follows: (i) type of ultrasound image, (ii) size of the ultrasound image 110, (iii) size of the target area of interest 120 (i.e. portion of the tissue and filter out the overlying dermis, underlying muscle, and muscle fascia), and (iv) annotated descriptor 130 for the region of the human body being examined. Examples of these data labels applied to an ultrasound image are shown in FIG. 9. The aforementioned data labels applied to the training dataset are applied outside of the model by a physician and this input data set was separated into a training set of images and a validation set of images. The image classification engine may include any of the object detection models previously described.

The training set of images are then fed into the model over multiple epochs to predict the bounding boxes for the ultrasound image with associated data labels. As a result, the neural network provides a predicted output with its own corresponding labels (which are the same labels as shown in FIG. 9). After each epoch, the model is evaluated on the validation set using precision, recall, with a mean average precision at Intersection of Union (IoU)=0.50 (mAP@0.5). The weights of the neural network are adjusted to minimize the difference between the predicted output and the actual label of each image in the training dataset 100 until the desired accuracy is reached based on IoU, precision and recall for each label. For the ultrasound image region (shown as 110), detections were considered correct if IoU≥0.90 with the ground-truth bounding box or within +8 pixels of the true edges, and precision and recall was ≥0.995 for this label. For physician depth markers (shown as 120), which are small and narrow, detections were considered correct if IoU≥0.50 or if the predicted centerline was within 10 pixels of ground truth, and precision and recall was ≥0.98. For text annotations (shown as 130), detections were considered correct if IoU≥0.50, and precision and recall was ≥0.97. These steps are repeated until the image classification engine can predict each label of new, unseen ultrasound images with a high degree of accuracy. Dataset expansion stopped once these thresholds were consistently achieved, and once the image-level pass rate—defined as all three required classes present and correct within an image—reached ≥0.99 for multiple consecutive validation epochs. Random seeds were fixed, and training metadata (dataset manifest hash, commit hash of training code, and model version) were captured in the CI pipeline to ensure reproducibility.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the system (and components of the individual operating components of the system) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

What is claimed is:

1. A computer-implemented method of training a neural network for recognizing a superficial fascial system from a patient ultrasound image comprising:
providing a training dataset comprising a plurality of ultrasound training images, each of the plurality of ultrasound training images comprising a set of data labels;
providing a validation dataset comprising a plurality of ultrasound training images, each of the plurality of ultrasound training images comprising a set of data labels;
initializing a convolutional neural network configured according to an image classification model architecture, the convolutional neural network comprising a plurality of convolutional layers, feature extraction layers, and output layers for predicting bounding boxes;
feeding the training dataset to the convolutional neural network to generate a predicting bounding box for each data label,
comparing the predicting bounding boxes of the training dataset to the bounding boxes of the validation dataset to determine a validation accuracy metric; and
updating the neural network parameters until the validation accuracy metric satisfies a predetermined convergence criterion,
wherein the resulting trained neural network model is configured to perform object detection on the patient ultrasound image in real time.

2. The method of claim 1, wherein the data labels are (i) type of ultrasound image, (ii) size of the ultrasound image, (iii) the target area, and (iv) the region of the human body being examined.

3. The method of claim 2, wherein the loss function further comprises an Intersection-over-Union (IoU) based penalty term for bounding box overlap.

4. The method of claim 1, wherein the neural network model is a You Only Look Once (YOLO) model.

5. The method of claim 4, wherein the neural network model architecture further comprises a path aggregation network (PANet) configured to enhance multi-scale feature fusion.

6. A system for determining the strength of a superficial fascial system of a patient, comprising:
an ultrasonic imaging system operable to generate an ultrasound image of a portion of subcutaneous tissue of the patient;
a processing module in communication with the ultrasonic imaging system, wherein said processing module comprises an image classification engine, said image classification engine being trained to identify the superficial fascial system and export a target area of the superficial fascial system into a second image,
wherein the processor further compares the total echogenicity of each pixel in the second image to the total number of pixels in the second image to determine a mean gray value for the superficial fascial system.

7. The system of claim 6, wherein the image classification engine is operable to identify a set of data labels in the ultrasound image.

8. The system of claim 7, wherein the data labels are (i) type of ultrasound image, (ii) size of the ultrasound image, (iii) the target area, and (iv) the region of the human body being examined.

9. A method for reducing complications for a surgical incision, said method comprising:
collecting an ultrasound image of a portion of subcutaneous tissue prior to the patient undergoing a surgical procedure;
identifying a target area of the subcutaneous tissue, the target area being defined as a portion of the ultrasound that excludes portions of the ultrasound image pertaining to the overlying dermis, underlying muscle, and muscle fascia;
determining a mean gray value for the target area; and
if the mean gray value is less than 0.127, recommending procedures to reduce tension at the surgical incision.

10. The method of claim 9 wherein a recommended procedure comprises removing excess skin at the surgical incision such that opposing skin flaps lay in apposition prior to final closure.

11. The method of claim 9 wherein a recommended procedure comprises adjusting either the posture or position of a patient to reduce tension at the surgical incision.

12. The method of claim 9 wherein a recommended procedure comprises utilizing a device operable to reduce tension at the surgical incision.

13. The method of claim 12 wherein the device comprises a negative-pressure vacuum device.
